(19) United States
(12) Patent Application Publication
(10) Pub. No.: US 2004/0230176 A1
(43) Pub. Date: Nov. 18, 2004

(54) System for treating a vascular condition that inhibits restenosis at stent ends

(75) Inventors: John P. Shanahan, Santa Rosa, CA (US); John E. Nolting, Santa Rosa, CA (US); Kevin M. Mauch, Windsor, CA (US); Zachary J. Woodson, Windsor, CA (US)

Correspondence Address: MEDTRONIC VASCULAR, INC., IP LEGAL DEPARTMENT, 3576 UNOCAL PLACE, SANTA ROSA, CA, 95403, US

(73) Assignee: Medtronic Vascular, Inc., Santa Rosa, CA

(21) Appl. No.: 10/828,969

(22) Filed: Apr. 21, 2004

Related U.S. Application Data
(60) Provisional application No. 60/464,724, filed on Apr. 23, 2003.

(51) Int. Cl. A61M 31/00

(52) U.S. Cl. 604/509; 604/103.01; 623/1.42; 623/1.1

(57) ABSTRACT

The present invention provides a system for treating a vascular condition that inhibits restenosis at the ends of a stent by delivering a therapeutic agent to the vessel wall at and beyond the stent ends. The system comprises a catheter, a stent, and a therapeutic agent. The catheter includes an inflatable balloon. The stent is operably coupled to the balloon. The therapeutic agent is disposed on at least a portion of the balloon and is delivered to a wall of a vessel at a proximal and a distal end of the stent when the balloon is inflated. The therapeutic agent may also be disposed on at least a portion of the stent. The system may further comprise a protective coating disposed under the therapeutic agent, over the therapeutic agent, or both under and over the therapeutic agent.

### RELATED APPLICATION

This application claims priority to U.S. Provisional Application No. 60/464,724, “System for Treating a Vascular Condition that Inhibits Restenosis at Both Ends” to John P. Shanahan et al., filed Apr. 23, 2003, the entirety of which is incorporated by reference.

### TECHNICAL FIELD

This invention relates generally to biomedical devices that are used for treating vascular conditions. More specifically, the invention relates to a system for treating a vascular condition that inhibits restenosis at stent ends.

### BACKGROUND OF THE INVENTION

Stents are generally cylindrical-shaped devices that are radially expandable to hold open a segment of a vessel or other anatomical lumen after implantation into the lumen. Various types of stents are in use, including expandable and self-expanding stents. Expandable stents generally are conveyed to the area to be treated on balloon catheters or other expandable devices. For insertion, the stent is positioned in a compressed configuration along the delivery device, for example crimped onto a balloon that is folded or otherwise wrapped about a guide wire that is part of the delivery device. After the stent is positioned across the lesion, it is expanded by the delivery device, causing the diameter of the stent to expand. For a self-expanding stent, commonly a sheath is retracted, allowing expansion of the stent.

Stents are used in conjunction with balloon catheters in a variety of medical therapeutic applications, including intravascular angioplasty. For example, a balloon catheter device is inflated during percutaneous transluminal coronary angioplasty (PTCA) to dilate a stenotic blood vessel. The stenosis may be the result of a lesion such as a plaque or thrombus. When inflated, the pressurized balloon exerts a compressive force on the lesion, thereby increasing the inner diameter of the affected vessel. The increased interior vessel diameter facilitates improved blood flow.

Soon after the procedure, however, a significant proportion of treated vessels restenose. To prevent restenosis, a stent, constructed of a metal or polymer, is implanted within the vessel to maintain lumen size. The stent acts as a scaffold to support the lumen in an open position. Configurations of stents include a cylindrical tube defined by a solid wall, a mesh, interconnected stents, or like segments. Exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau.

Stent insertion may cause undesirable reactions such as inflammation, infection, thrombosis, and proliferation of cell growth that occludes the passageway. Therapeutic agents that assist in preventing these conditions have been delivered to the site by coating these agents onto a stent. However, this can result in drug being delivered to only those portions of the vessel in direct contact with the stent. Because restenosis is often a greater problem in tissue just beyond the ends of the stent than it is in the tissue at least partially opposed by the stent, drug delivery using the stent alone may not be fully effective.

Therefore, it would be desirable to have a system for treating a vascular condition that overcomes the aforementioned and other disadvantages.

### SUMMARY OF THE INVENTION

One aspect of the present invention is a system for treating a vascular condition, comprising a catheter, an inflatable balloon, a stent, and a therapeutic agent. The stent is operably coupled to the balloon. A therapeutic agent is disposed on at least a portion of the balloon and is delivered to a wall of a vessel at a proximal and a distal end of the stent when the balloon is inflated within the vessel.

Another aspect of the present invention is a method of manufacturing a system for treating a vascular condition. A catheter is provided, the catheter including an inflatable balloon. A stent is coupled to the catheter. A therapeutic agent is applied to at least a portion of the balloon. The therapeutic agent is delivered to a wall of a vessel at a proximal and a distal end of the stent when the balloon is inflated within the vessel.

Yet another aspect of the present invention is a method of inhibiting restenosis at the ends of a stent used to treat a vascular condition. A site is identified for treatment. A stent is introduced into a vessel containing the site identified for treatment. The stent is coupled to a catheter, the catheter including an inflatable balloon with a therapeutic agent disposed on at least a portion of the balloon. The stent is guided to a position adjacent the site identified for treatment. The balloon is expanded. Expanding the balloon delivers the stent and contacts a wall of the vessel with the balloon such that the therapeutic agent disposed on the balloon is delivered to the wall of the vessel at a proximal and a distal end of the stent. The balloon is deflated. The catheter is withdrawn from the vessel.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a system for treating a vascular condition, in accordance with the present invention;

FIG. 2 is a flow diagram of one embodiment of a method of manufacturing a system for treating a vascular condition, in accordance with the present invention;

FIG. 3 is a flow diagram of another embodiment of a method of manufacturing a system for treating a vascular condition, in accordance with the present invention; and

FIG. 4 is a flow diagram of one embodiment of a method of inhibiting restenosis at the ends of a stent used to treat a vascular condition, in accordance with the present invention.

### DETAILED DESCRIPTION OF THE PRESENTLY PREFERRED EMBODIMENTS

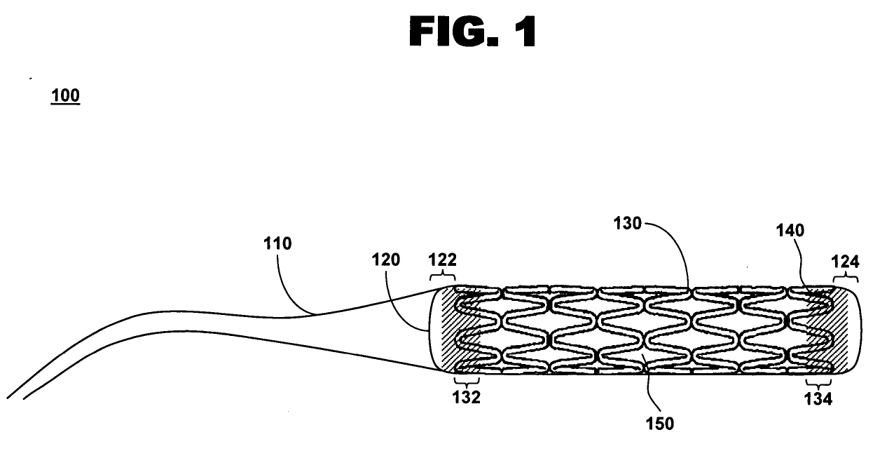

One aspect of the present invention is a system for treating a vascular condition. One embodiment of the system, in accordance with the present invention, is illustrated in FIG. 1 at 100. System 100 comprises a catheter 110, an inflatable balloon 120, a stent 130, a therapeutic agent 140, and a protective coating 150. Balloon 120 includes a proximal portion 122 and a distal portion 124. Stent 130 includes a proximal portion 132 and a distal portion 134.

Catheter 110 may be any catheter known in the art that is capable of inflating a balloon and appropriate for delivering a stent to a treatment site, for example a percutaneous transluminal coronary angioplasty (PTCA) balloon catheter having a low profile design with a tapered distal tip, an inner lumen for insertion of a conventional guide wire, and a hollow tubular portion that is in communication with a source of inflation. Catheter 110 includes an inflatable balloon 120.

Balloon 120 may be made of a suitable material such as polyethylene, polyethylene terephthalate (PET), nylon, or the like. Balloon 120 includes a proximal portion 122 and a distal portion 124. The dimensions of balloon 120 may be selected based on the dimensions of the stent being delivered by the system.

Stent 130 may comprise a variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above. Stent 130 is operably coupled to balloon 120. In the present embodiment, balloon proximal portion 122 extends beyond a proximal end of the stent, and balloon distal portion 126 extends beyond a distal end of the stent. Each balloon portion may extend beyond the stent a distance of approximately 0.5 to 1 millimeter, for example. Alternatively, the balloon may extend beyond the proximal and distal ends of the stent only after it has been expanded within the vessel.

Therapeutic agent 140 is disposed on at least a portion of balloon 120 and is delivered to a wall of a vessel when balloon 120 is inflated within the vessel. Therapeutic agent 140 is intended to inhibit restenosis at the ends of the stent and may include, for example, an antiproliferative agent, an antineoplastic agent, an antibiotic agent, an anti-inflammatory agent, and combinations thereof. Therapeutic agent 140 may be lipophilic. Lipophilic agents are rapidly absorbed by the fatty tissue of a vessel wall and may, therefore, be delivered to the vessel wall by even a brief inflation of the balloon, for example when the balloon is inflated and in contact with the vessel wall for just 30 to 60 seconds. The pressures exerted on the vessel wall by the inflated balloon may contribute to efficient delivery of the therapeutic agent.

Therapeutic agent 140 may also be disposed on at least a portion of stent 130. As seen in FIG. 1, therapeutic agent 140 is disposed on the distal and proximal portions of both the balloon and the stent. However, one skilled in the art will recognize that the therapeutic agent may be disposed on at least a portion of one or both of the balloon and the stent in a variety of configurations, having been applied either before or after coupling the stent to the balloon.

Protective coating 150 may be disposed on all or a portion of at least one of balloon 120, stent 130, and therapeutic agent 140. It may be disposed under the therapeutic agent, over the therapeutic agent, or both under and over the therapeutic agent. In the present embodiment, the protective coating is disposed on the entire stent outer surface and those portions of the balloon not covered by the stent, having been applied as an overspray prior to applying the therapeutic agent. This protective coating may not only protect the balloon and stent, but also serve as a primer coating for the therapeutic agent. In the present embodiment, the protective coating is additionally disposed on at least those portions of the balloon and stent carrying therapeutic agent 140, serving to protect the therapeutic agent from damage during storage and shipment, as well as during delivery to a treatment site within a vessel.

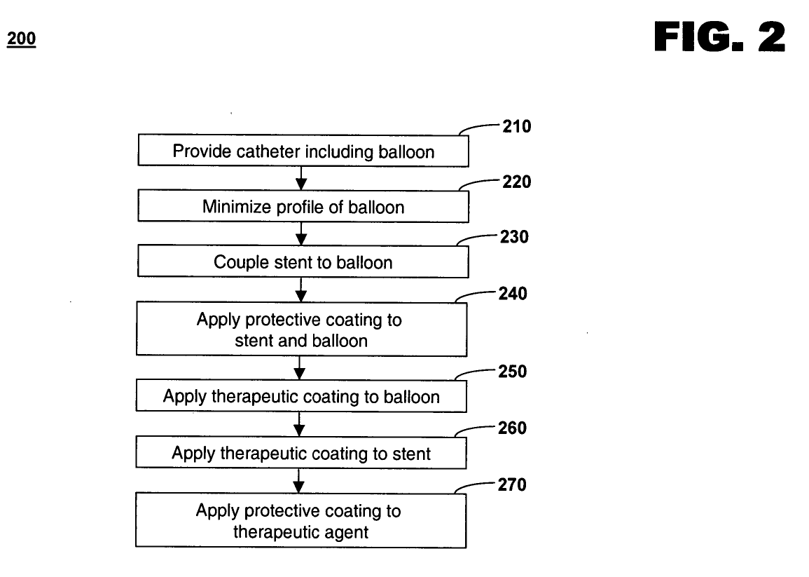

A further aspect of the present invention is a method of manufacturing a system for treating a vascular condition. FIG. 2 shows a flow diagram of one embodiment, in accordance with the present invention at 200.

A catheter is provided, the catheter including an inflatable balloon (Block 210). The catheter may be any catheter known in the art that is capable of inflating a balloon and appropriate for delivering a stent to a treatment site. The balloon may be made of a suitable material such as polyethylene, polyethylene terephthalate (PET), nylon, or the like.

The balloon is folded or otherwise manipulated or treated to minimize its profile (Block 220). A stent is then coupled to the balloon such that a proximal portion of the balloon extends beyond a proximal end of the stent and a distal portion of the balloon extends beyond a distal end of the stent (Block 230). The balloon may extend beyond the stent approximately 0.5 to 1 millimeter, for example, in either direction.

The stent may comprise a variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above.

A protective coating is applied to the stent and the portions of the balloon not covered by the stent (Block 240). The protective coating may be applied by, for example, spraying the coupled stent and balloon with the coating.

A therapeutic agent is applied to the proximal and distal portions of the balloon that extend beyond the stent (Block 250). The therapeutic agent is intended to inhibit restenosis at the ends of the stent and may include, for example, an antiproliferative agent, an antineoplastic agent, an antibiotic agent, anti-inflammatory agent, and combinations thereof. The therapeutic agent may be lipophilic. A method such as infusing, dipping, spraying, pad printing, inkjet printing, rolling, painting, micro-spraying, wiping, electrostatic deposition, vapor deposition, epitaxial growth, and combinations thereof may be used to apply the therapeutic agent to the balloon.

At the same time the therapeutic agent is applied to proximal and distal portions of the balloon, it may also be applied to proximal and distal portions of the stent (Block 260). The protective coating is then applied again, this time over at least the therapeutic agent (Block 270). This coating may aid in maintaining the therapeutic agent on the balloon and stent until it is delivered to the wall of a vessel by the balloon being inflated within the vessel.

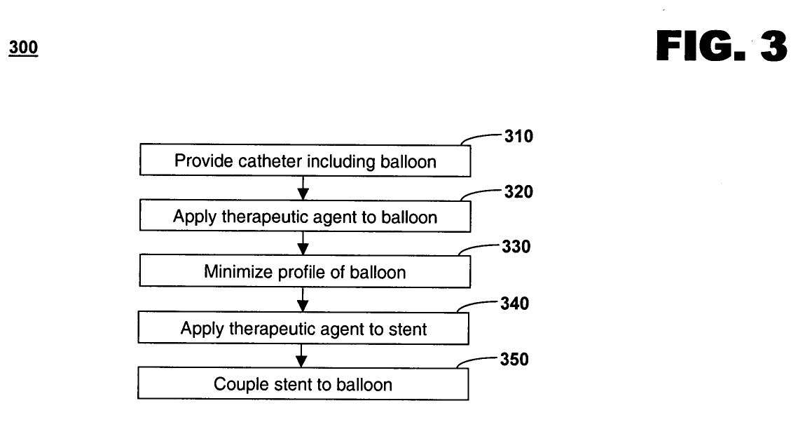

FIG. 3 shows a flow diagram of another embodiment of a method of manufacturing a system for treating a vascular condition, in accordance with the present invention at 300.

A catheter is provided, the catheter including an inflatable balloon (Block 310). The catheter may be any catheter known in the art that is capable of inflating a balloon and appropriate for delivering a stent to a treatment site. The balloon may be made of a suitable material such as polyethylene, polyethylene terephthalate (PET), nylon, or the like.

A therapeutic agent is applied to at least a portion of the balloon, for example to distal and proximal ends of the balloon (Block 320). The therapeutic agent is intended to inhibit restenosis and may include, for example, an antiproliferative agent, an antineoplastic agent, an antibiotic agent, an anti-inflammatory agent, and combinations thereof. The therapeutic agent may be lipophilic. A method such as infusing, dipping, spraying, pad printing, inkjet printing, rolling, painting, micro-spraying, wiping, electrostatic deposition, vapor deposition, epitaxial growth, and combinations thereof may be used to apply the therapeutic agent to the balloon. The balloon is then folded or otherwise manipulated or treated to minimize its profile (Block 330).

The therapeutic agent is applied to at least a portion of a stent, for example to the distal and proximal portions of the stent (Block 340). The stent may comprise a variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above.

The therapeutic agent may be applied to the stent using a method that is the same as or different from that used to apply the therapeutic agent to the balloon. In this embodiment, the therapeutic agent is applied to the stent before the stent is coupled to the balloon. Applying the therapeutic agent to the balloon and the stent prior to coupling the stent to the balloon may provide better coverage of both the stent and the balloon.

The stent is then coupled to the balloon (Block 350). The stent may be coupled to the balloon such that a proximal portion of the balloon extends beyond a proximal end of the stent and a distal portion of the balloon extends beyond a distal end of the stent. Alternatively, the balloon may be formed or folded in such a way that it extends beyond the proximal and distal ends of the stent when the balloon is expanded. In either case, the therapeutic agent is delivered to the wall of a vessel when the balloon is inflated within the vessel.

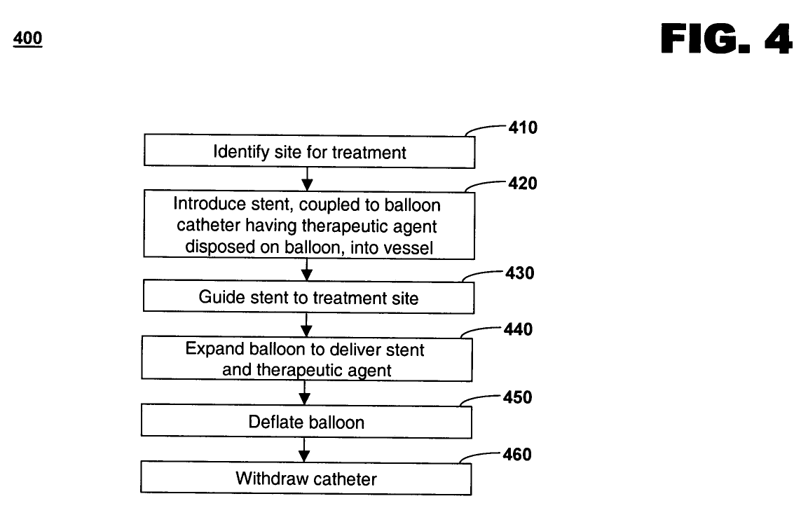

Yet another aspect of the present invention is a method of inhibiting restenosis at the ends of a stent used to treat a vascular condition. FIG. 4 shows a flow diagram of one embodiment, in accordance with the present invention at 400.

A site is identified for treatment (Block 410). This may be accomplished using a conventional method such as angiography. A stent is then introduced into a vessel containing the site, the stent coupled to a catheter, the catheter including an inflatable balloon with a therapeutic agent disposed on at least a portion of the balloon (Block 420). The stent may be introduced into the vessel by, for example, creating a percutaneous access site in the vessel to be treated or a vessel that leads to the vessel to be treated.

The stent is guided to a position adjacent the site identified for treatment (Block 430). This may be accomplished by introducing a guide wire through the percutaneous access site and advancing the stent coupled to the catheter over the guide wire to a position adjacent to the site identified for treatment.

The balloon is expanded by, for example, introducing a fluid into the balloon through a lumen within the catheter, thereby pressurizing and expanding the balloon (Block 440). Expanding the balloon delivers the stent and contacts a wall of the vessel with the balloon such that the therapeutic agent disposed on the balloon is delivered to the wall of the vessel at a proximal and a distal end of the stent.

The therapeutic agent is intended to inhibit restenosis at the ends of the stent and may include, for example, an antiproliferative agent, an antineoplastic agent, an antibiotic agent, an anti-inflammatory agent, and combinations thereof. The therapeutic agent may be lipophilic. Lipophilic agents are rapidly absorbed by the fatty tissue of a vessel wall and may, therefore, be delivered to the vessel wall by even a brief inflation of the balloon, for example when the balloon is inflated and in contact with the vessel wall for just 30 to 60 seconds. The pressures exerted on the vessel wall by the inflated balloon may contribute to efficient delivery of the therapeutic agent.

The balloon is deflated (Block 450). The catheter is then withdrawn from the vessel (Block 460).

In practice, the present invention inhibits restenosis at the ends of a stent by delivering a therapeutic agent to the vessel wall at and beyond the stent ends.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A system for treating a vascular condition, comprising:
a catheter, the catheter including an inflatable balloon;
a stent operably coupled to the balloon; and
a therapeutic agent disposed on at least a portion of the balloon, wherein the therapeutic agent is delivered to a wall of a vessel at a proximal and a distal end of the stent when the balloon is inflated within the vessel.

2. The system of claim 1 wherein the therapeutic agent inhibits restenosis at the ends of the stent.

3. The system of claim 1 wherein the therapeutic agent includes at least one agent selected from a group consisting of an antiproliferative agent, an antineoplastic agent, an antibiotic agent, and an anti-inflammatory agent.

4. The system of claim 1 wherein the therapeutic agent is lipophilic.

5. The system of claim 1 wherein the therapeutic agent is further disposed on at least a portion of the stent.

6. The system of claim 5 wherein the therapeutic agent is disposed on at least a distal portion of the stent and a proximal portion of the stent.

7. The system of claim 1 further comprising:
a protective coating disposed on at least a portion of at least one of the balloon, the stent, and the therapeutic agent.

8. The system of claim 5 further comprising:
a protective coating disposed on at least a portion of at least one of the balloon, the stent, and the therapeutic agent.

9. A method of manufacturing a system for treating a vascular condition, comprising:
providing a catheter, the catheter including an inflatable balloon;
coupling a stent to the balloon; and
applying a therapeutic agent to at least a portion of the balloon, wherein the therapeutic agent is delivered to a wall of a vessel at a proximal and a distal end of the stent when the balloon is inflated within the vessel.

10. The method of claim 9 wherein the therapeutic agent inhibits restenosis at the ends of the stent.

11. The method of claim 9 wherein applying a therapeutic agent comprises applying the therapeutic agent to at least a portion of the balloon prior to coupling the stent to the balloon.

12. The method of claim 9 wherein coupling a stent to the balloon comprises coupling the stent such that a proximal portion of the balloon extends beyond a proximal end of the stent and a distal portion of the balloon extends beyond a distal end of the stent.

13. The method of claim 9 wherein applying a therapeutic agent comprises applying the therapeutic agent to at least the proximal and distal portions of the balloon after coupling the stent to the balloon.

14. The method of claim 9 further comprising:
applying the therapeutic agent to at least a portion of the stent.

15. The method of claim 14 wherein applying a therapeutic agent to at least a portion of the stent comprises applying the therapeutic agent prior to coupling the stent to the balloon.

16. The method of claim 14 wherein applying a therapeutic agent to at least a portion of the stent comprises applying the therapeutic agent after coupling the stent to the balloon.

17. The method of claim 14 wherein applying the therapeutic agent to at least a portion of the stent comprises applying the therapeutic agent to at least a proximal portion of the stent and a distal portion of the stent.

18. The method of claim 9 further comprising:
applying a protective coating over at least a portion of at least one of the balloon and the stent prior to applying the therapeutic agent.

19. The method of claim 9 further comprising:
applying a protective coating over at least a portion of at least one of the balloon, the stent, and the therapeutic agent.

20. The method of claim 14 further comprising:
applying a protective coating over at least a portion of at least one of the balloon, the stent, and the therapeutic agent.

21. The method of claim 9 further comprising:
minimizing the balloon profile prior to coupling the stent to the balloon.

22. A method of inhibiting restenosis at the ends of a stent used to treat a vascular condition, comprising:
identifying a site for treatment;
introducing a stent into a vessel containing the site identified for treatment, the stent coupled to a catheter, the catheter including an inflatable balloon, a therapeutic agent disposed on at least a portion of the balloon;
guiding the stent to a position adjacent the site identified for treatment;
expanding the balloon, wherein expanding the balloon includes delivering the stent and contacting a wall of the vessel with the balloon such that the therapeutic agent disposed on the balloon is delivered to the wall of the vessel at a proximal and a distal end of the stent;
deflating the balloon; and
withdrawing the catheter from the vessel.

23. The method of claim 22 wherein the therapeutic agent inhibits restenosis at the ends of the stent.

24. The system of claim 22 wherein the therapeutic agent includes at least one agent selected from a group consisting of an antiproliferative agent, an antineoplastic agent, an antibiotic agent, and an anti-inflammatory agent.

25. The system of claim 22 wherein the therapeutic agent is lipophilic.
